(19) United States
(12) Patent Application Publication
(10) Pub. No.: US 2023/0274420 A1
(43) Pub. Date: Aug. 31, 2023

(54) METHOD AND SYSTEM FOR AUTOMATED GENERATION OF TEXT CAPTIONS FROM MEDICAL IMAGES

(71) Applicant: Harrison-AI Pty Ltd., Sydney, New South Wales (AU)

(72) Inventors: Jarrel Seah, Sydney, New South Wales (AU); Xavier Holt, Sydney (AU)

(21) Appl. No.: 18/015,036

(22) PCT Filed: Jun. 28, 2021
(86) PCT No.: PCT/AU2021/050685

§ 371 Date: Jan. 6, 2023

(30) Foreign Application Priority Data
Jul. 6, 2020 (AU) 2020902318
Mar. 31, 2021 (AU) 2021900946

(51) Int. Cl. G06T 7/00; G16H 30/40

(52) U.S. Cl. CPC G06T 7/0012; G16H 30/40; G06T 2207/20081

(57) ABSTRACT

Computer implemented method for generating captions for medical images and/or clinical reports are provided. The methods comprise obtaining one or more medical images; using an image processing component to process the one or more images, wherein the image processing component comprises a deep learning model that takes as input the one or more medical images and produces as an output an image feature tensor; and using a natural language processing component to generate a caption for the one or more medical images, wherein the natural language processing component comprises a transformer-based model that takes as input the image feature tensor from the image processing component and produces as output a probability for each word in a vocabulary. Related systems and products are also described.

### CROSS REFERENCE TO RELATED APPLICATIONS

This application is a National Stage Application filed under 35 U.S.C. § 371 of International Patent Application No. PCT/AU2021/050685, filed Jun. 28, 2021, which claims priority to Australian patent application 2021900946, filed on Mar. 31, 2021, which claims priority to Australian patent application 2020902318 Jul. 6, 2020, the contents of each of which are incorporated herein by reference in their entireties.

### FIELD OF THE INVENTION

The present invention relates generally to a method and system for automated generated of text captions from medical images.

### BACKGROUND OF THE INVENTION

Interpreting and summarising the information contained in medical images such as e.g. histopathology images or radiography images is time consuming and to date requires trained experts. It would be highly beneficial to develop automated methods that can provide a textual description of such images.

### SUMMARY OF THE INVENTION

Pertinent information about medical images is often described and summarized in text form for categorization and communication. The present inventors have identified that utilizing these paired datasets of images and text captions, it was possible to construct a hybrid artificial intelligence (AI) model that generates plausible text captions, and in the process learns useful information about the images that can be used in downstream tasks like image classification or object detection. The present inventors have also identified that such a model could be configured to ‘autocomplete’ reports given some seed text, as well as measure the perplexity of an image caption given a paired image. For instance, given one or more input images and some seed text, a user could be offered suggestions on plausible reports conditioned on the inputs, enabling them to quickly complete their report by agreeing or modify the text by continuing to type. Such a model can further be fine-tuned to prior examples of the user's reports, enabling generation of user-specific text. Parameters for the user to define include the length of the report suggestion as well as the ‘temperature’ of the suggestions where ‘hotter’ suggestions are more unique but less likely overall to occur. The temperature is a setting set by each user. This setting can be adjusted by its user to change the type of reports that are generated. By increasing the ‘heat’ level of this setting, the algorithm tends to generate words that are less likely to occur.

Anticipated use cases for the method and system provided by the present invention include: generating reports from histopathology data (which may include e.g. images of histopathology slides), generating reports from macroscopic ‘gross pathology’ specimen data (which may include e.g. images of gross pathology specimens such as organs, tissues, body cavities, etc.), generating reports from radiology images, generating figure captions for journal articles (such as e.g. figures including medical images of any of the above-mentioned type).

Compared to solutions such as that of Biswall et al., the system disclosed herein is trained from end-to-end without having to index past sentences. This enables the present system to generate completely novel sentences which may not exist in any prior corpus, rather than simply editing sentences from previous reports. Further, the present invention improves upon the prior art by using a transformer-based model which is a self-attention model (relying entirely on self-attention to compute representations of its input and output without using sequence-aligned RNNs or convolution), to perform sequence transduction which can attend to any previous part of the sentence with equal ease, and hence can capture long range dependencies more effectively compared to the recurrent neural networks used in the prior art.

When decoding with a transformer model the image features are available without degradation at every step. It is, accordingly, an object of at least one embodiment of the present invention to address the need for better tools to automatically generate text captions from medical images, and in particular to provide tools that can do this in a fast and accurate manner. It is an object of at least one embodiment of the present invention to be highly parallelisable to train and at a significantly reduced number of FLOPs (floating-point operations).

Accordingly, in one aspect, there is provided a computer implemented method for generating captions for medical images, the method comprising: obtaining one or more medical images; using an image processing component to process the one or more images, wherein the image processing component comprises a deep learning model that takes as input the one or more medical images and produces as an output an image feature tensor; and using a natural language processing component to generate a caption for the one or more medical images, wherein the natural language processing component comprises a transformer-based model that takes as input the image feature tensor from the image processing component and produces as output a probability for each word in a vocabulary.

Using a natural language processing component to generate a caption for the one or more medical images may comprise (i) using the transformer-based model to predict a probability for each word in the vocabulary and (ii) sampling one or more words using the probabilities from step (i). Using a natural language processing component to generate a caption for the one or more medical images may further comprise repeating steps (i) and (ii) for one or more iterations, wherein a next word is predicted at each iteration.

The transformer-based model may further takes as input a tensor derived from a set of one or more words. As such, the method may further comprise obtaining seed text, and the one or more words may comprise the seed text.

The method may further comprise repeating steps (i) and (ii) for one or more iterations, wherein the one or more words comprise the words generated at any preceding iterations. The number of iterations may be derived from a predetermined text length. The method may comprise receiving a predetermined text length from a user.

The method may further comprise obtaining the tensor derived from a set of one or more words by tokenising and embedding a set of one or more words. The tokenising is advantageously performed using byte pair encoding. The embedding may be performed using an embedding algorithm comprising a lookup table where each input token is mapped to a vector of size M (where M is the size of the embedding used by the transformer-based model). The embedding algorithm may comprise one or more parameters such as the values in the lookup table, which may be optimised during training of the natural language processing component. The vocabulary for the embedding algorithm may be learned in an unsupervised fashion from domain-specific text, in order to allow for more efficient encoding, training and caption-generation.

The deep learning model may be a convolutional neural network (CNN). The deep learning model may be obtained by training a pre-trained CNN model, A convenient pre-trained CNN model is an EfficientNet model, such as EfficientNet-B0. In another embodiment, the pre-trained CNN model may be a DenseNet model such as Densenet-121. The CNN model may also be pre-trained on a domain-specific task, such as image classification and segmentation in medical images.

The transformer-based model may be obtained by training a pre-trained GPT-2 model, a pre-trained BERT model or a pre-trained T5 model. The transformer-based model is preferably a GPT-2 model.

Notably, the transformer-based model may also instead be one of a family of sub-quadratic (sometimes linear) complexity models. This may take the form of the Reformer model, the Linformer model or models described in the papers ‘Transformers are RNNs: Fast Autoregressive Transformers with Linear Attention’, or ‘Fast Transformers with Clustered Attention’. By doing so we can train on and generate longer captions and improve training and decoding efficiency.

The image processing component and the natural language processing component may have been trained jointly. The components may have been trained jointly to minimise the cross-entropy loss and/or the perplexity of the predictions of the transformer-based model over a set of data. Secondary objectives and loss functions may also be defined, such as an image-classification loss on the output of the image processing component; or a text-classification loss on the output of the transformer-based model. These losses are highly variable. These secondary objectives may be trained in a single pass of the data through the system, and their loss may be weighted and combined with the primary loss. Furthermore, joint training may also be made more efficient by generating captions at training time and using them to enforce a non-differentiable loss function via reinforcement learning.

Jointly training the image processing component and the natural language processing component may comprise optimising one or more parameters of the image processing component and one or more parameters of the natural language processing component simultaneously. The parameters of the image processing component may comprise one or more parameters of the deep neural network. The parameters of the natural language processing component may comprise one or more parameters of the transformer-based model. The parameters of the natural language processing component may comprise one or more parameters of the text embedding algorithm.

The method may further comprise receiving training data from a user and at least partially re-training the models in the image processing component and the natural language processing component using the training data received from the user. The training data received from the user may comprise training seed text associated with one or more training images or training seed text associated with one or more training images and the one or more training images.

The one or more images may comprise multiple images and the method may comprise generating a caption for the multiple images jointly. The multiple images are preferably related to each other by sharing one or more features selected from: being associated with the same subject, being acquired using the same modality, showing the same pathology, showing the same organ or body part.

The image processing component and the natural language processing component may have been trained using training data comprising images that share one or more features with the one or more images, the one or more features being selected from: being associated with the same subject, being acquired using the same modality, showing the same pathology, showing the same organ or body part. The training data may also be preferably comprised of images that share one or more features with an associated caption, such as: being associated with the same subject, describing the same case, or representing the same clinical finding.

The method may further comprise pre-processing the one or more images. Pre-processing may refer to any step applied prior to the processing by the image analysis component. Examples of pre-processing steps that can be applied to the one or more images comprise performing one or more steps selected from: randomly re-ordering the images (i.e. such that the images are input to the image analysis component in a different order from the order in which they were obtained), normalising pixel values across multiple images, changing the aspect ratio of one or more images (e.g. to a common aspect ratio across multiple images), scaling the one or more images (e.g. to a common scale across multiple images), re-sizing the one or more images (e.g. to a common size across multiple images).

The caption may comprise free text. The one or more medical images may be associated with a patient and the caption may be a clinical report for the patient. The one or more medical images may be selected from: histopathology images, radiography images, magnetic resonance images, ultrasound images, endoscopy images, positron emission tomography (PET) images, single-photon emission computed tomography (SPECT) images, and gross pathology images. The one or more medical images may also be arranged such that a given subject's relevant clinical history is provided as context for generating the caption.

The natural language processing component may comprise a transformer-based model with a single stack architecture.

The transformer-based model may use an attention mask. The attention mask may be configured to forbid elements in an input tensor derived from a set of one or more words from attending to one another. For example, the attention mask may be configured such that the transformer-based model generates words in an auto-regressive way. Some attention masks may be configured to randomly mask a predetermined proportion of the input tensor derived from a set of one or more words.

The transformer-based model preferably comprises one or more encoder and zero or more decoder blocks, each comprising a multi-head attention layer.

The image processing component may be configured to produce an image feature tensor of size N×M, wherein M is the size of the embedding used by the transformer-based model and N is the number of images in the one or more images.

The input tensor derived from one or more images may be generated on a per-image basis by dividing the spatially aware feature-map into a grid, and pooling within a grid to generate a fixed-length vector. The pooling can be done either via max or average pooling. Max pooling means that for each patch, the maximum per dimension is taken in the feature vector, and then average the vectors in each patch. The resulting tensor for each image will be of shape G×M, where G is the number of cells in the subdivision grid which can be one or more and M is the number of channels in the resulting feature map.

The input tensor derived from one or more words has a size K×M, wherein M is the size of the embedding used by the transformer-based model and K is the number of tokens derived from the one or more words by tokenisation. These input tensors may be intelligently and dynamically batched together such that tensors of similar length are batched together, and that the total number of elements in a batch is dependent on the length of the longest tensor in that batch. Intelligently and dynamically refers to the ability to select samples in a non-random fashion. Typically deep learning models are trained using random shuffling so each batch has a homogeneously selected random group of samples. Intelligently grouping the samples by selecting samples in a non-random fashion such that each batch is of a similar length reduces memory and compute resources. This relationship may not be linear—for example, a batch whose longest element is twice as long as another may have only one quarter of the elements.

The transformer-based model may take as input a tensor that comprises the image feature tensor pre-pended to the input tensor derived from the one or more words.

The transformer-based model may further take as input a vector comprising information about the relative position of elements in the input tensor derived from one or more words or input images, wherein the relative position of the elements corresponds to the order of the one or more words or input images from which the input tensor was derived.

The transformer-based model may comprise a plurality of encoder blocks and a plurality of decoder blocks. For example, the transformer-based model may comprise at least 12 blocks, such as e.g. at least 6 encoder blocks and 6 decoder blocks.

According to a further aspect, there is provided a computer implemented method for generating a clinical report for a patient, the method comprising: receiving one or more medical images associated with the patient; using an image processing component to process the one or more images, wherein the image processing component comprises a deep learning model that takes as input the one or more medical images and produces as an output an image feature tensor; and using a natural language processing component to generate a clinical report associated with the one or more medical images, wherein the natural language processing component comprises a transformer-based model that takes as input the image feature tensor from the image processing component and produces as output a probability for each word in a vocabulary.

The method of the present aspect may have any of the features of the previous aspect.

According to a further aspect, there is provided a computer implemented method for automatically completing a clinical report for a patient, the method comprising: receiving one or more medical images associated with the patient; receiving one or more words associated with the medical images and/or the patient; using an image processing component to process the one or more images, wherein the image processing component comprises a deep learning model that takes as input the one or more medical images and produces as an output an image feature tensor; using a natural language processing component to generate a clinical report associated with the one or more medical images, wherein the natural language processing component comprises a transformer-based model that takes as input the image feature tensor from the image processing component and a seed text tensor derived from the one or more words, and produces as output a probability for each word in a vocabulary.

Using a natural language processing component to generate a clinical report associated with the one or more medical images may comprise (i) using the transformer-based model to predict a probability for each word in the vocabulary and (ii) sampling one or more words using the probabilities from step (i). Using a natural language processing component to generate a clinical report may comprise repeating steps (i) and (ii) for one or more iterations, wherein a next word is predicted at each iteration. Using a natural language processing component to generate a clinical report may comprise repeating steps (i) and (ii) for one or more iterations, wherein a next word is predicted at each iteration. The method may further comprise repeating steps (i) and (ii) for one or more iterations, and the seed text tensor is derived from the one or more words and the words generated at any preceding iterations.

The method of the present aspect may have any of the features of the first aspect.

The methods of any preceding aspect may further comprise providing at least part of the output of the natural language processing component to a user via a user interface.

According to a further aspect, there is provided a computer implemented method for providing a tool. The tool may be configured to perform the method of any preceding aspect. The method of the present aspect comprises: obtaining a plurality of sets of training images, each set comprising one or more medical images; obtaining a plurality of training text each comprising one or more words associated with a respective set of training images; and jointly training a model comprising: an image processing component comprising a deep learning model that takes as input one or more medical images and produces as output an image feature tensor; and a natural language processing component comprising a transformer-based model that takes as input the image feature tensor from the image processing component and produces as output a probability for each word in a vocabulary.

Jointly training the model may comprise: (i) using the transformer-based model to predict a probability for each word in the vocabulary based at least in part on an image feature tensor derived from a set of training images and (ii) determining the probability of a corresponding word in the training text associated with the set of training data. At step (i) the transformer-based model may predict a probability for each word in the vocabulary based at least in part on an image feature tensor derived from a set of training images and a text tensor derived from one or more words associated with the set of training images.

Jointly training the model may comprise: obtaining the text tensor by tokenising and embedding one or more words associated with the set of training images. The tokenising may be performed using byte pair encoding. The embedding may be performed using an embedding algorithm comprising a lookup table where each input token is mapped to a vector of size M (where M is the size of the embedding used by the transformer-based model).

The method may further comprise defining the vocabulary using the training text. This may be performed by tokenising the training text and defining the vocabulary as the set of tokens represented in the training text.

The method may have any of the features of the preceding aspect. In particular, the deep learning model and the transformer-based model may have any of the features described in relation to embodiments of the first aspect.

Jointly training the model may comprise optimising one or more parameters of the image processing component and one or more parameters of the natural language processing component, wherein the optimisation criteria comprise minimising the cross entropy loss and/or the perplexity of the predictions of the transformer-based model over at least a subset of the sets of training images and associated training text.

Jointly training the model may further comprise optimising one or more parameters of the text embedding algorithm.

The method may further comprise receiving additional training data from a user and at least partially re-training the model in the image processing component, the natural language processing component or both using the training data received from the user. The further training data received from the user may comprise further training text associated with one or more training images or further training seed text associated with one or more further training images and the one or more further training images.

The training images may comprise images that share one or more features being selected from: being acquired using the same modality, showing the same pathology, showing the same organ or body part.

The training text is preferably consistent in that similar images are associated with text that has similar cognitive content. The method may comprise excluding training text that is not consistent.

The training text is preferably informative in that summarises or otherwise describes the training images that it is associated with or relevant features thereof. The method may comprise excluding training text that is not informative.

The method may further comprise pre-processing the one or more training images, wherein pre-processing refers to any step applied prior to the processing by the image analysis component. Pre-processing the one or more images may comprise performing one or more steps selected from: randomly re-ordering the images in a set, normalising pixel values across images in a set, changing the aspect ratio of one or more images in a set to a common aspect ratio, scaling one or more images in a set to a common scale, and re-sizing one or more images to a common size.

The caption may comprises free text. The one or more medical images in each set may be associated with a respective patient and the training text may be a clinical report for the patient. The one or more medical images in each set may be associated with a respective patient and the caption may be a clinical report for the patient.

The one or more training images may be selected from: histopathology images, radiography images, magnetic resonance images, ultrasound images, endoscopy images, positron emission tomography (PET) images, single-photon emission computed tomography (SPECT) images, and gross pathology images.

According to a further aspect, there is provided a system comprising: at least one processor; and at least one non-transitory computer readable medium containing instructions that, when executed by the at least one processor, cause the at least one processor to perform the operations of any embodiment of any preceding aspect.

The system may be for generating captions for medical images, for generating a medical report from one or more medical images associated with a patient, for automatically completing a medical report associated with one or more medical images, and/or for providing a tool for generating captions, such as medical reports, for one or more medical images.

According to a further aspect, there is provided a non-transitory computer readable medium containing instructions that when executed by at least one processor, cause the at least one processor to perform the operations of any embodiment of any of the first to third aspects.

According to a further aspect, there is provided a system for generating captions for medical images, the system comprising: an image acquisition module, configured to acquire one or more medical images; a processor configured to: receive the or each image from the image acquisition module, and perform the steps of any embodiment of the first aspect using the one or more images received from the image acquisition module.

The processor may further be configured to receive one or more words from a user and perform the steps of any embodiment of the first aspect using the one or more images received from the image acquisition module and the one or more words received from the user.

The processor may be further configured to provide at least part of the output of the natural language component to a user via a user interface. The at least part of the output may comprise one or more captions generated by the natural language component based on the one or more images and optionally the one or more words received from the user.

Further aspects, advantages, and features of embodiments of the invention will be apparent to persons skilled in the relevant arts from the following description of various embodiments. It will be appreciated, however, that the invention is not limited to the embodiments described, which are provided in order to illustrate the principles of the invention as defined in the foregoing statements and in the appended claims, and to assist skilled persons in putting these principles into practical effect.

### BRIEF DESCRIPTION OF THE DRAWINGS

Embodiments of the invention will now be described with reference to the accompanying drawings, in which like reference numerals indicate like features, and wherein:

FIG. 1 is a diagram illustrating an exemplary computing system in which embodiments of the present invention may be implemented;

FIG. 2 is a diagram illustrating the architecture of a natural language processing component of a hybrid AI model according to embodiments of the invention;

FIG. 3 is a diagram illustrating the architecture of an image processing component of a hybrid AI model according to a first embodiment of the invention;

FIG. 4 shows an example of the use of the present invention to automatically provide image captions for histopathology images; and

FIG. 5 is diagram illustrating the architecture of an image processing component of a hybrid AI model according to a second embodiment of the invention.

### DETAILED DESCRIPTION OF EMBODIMENTS

As described above, it would be highly beneficial to develop automated methods that can provide a textual description of medical images. Initiatives to foster the developments of such tools have been undertaken, including the ImageCLEF competition, see e.g. Eickhoff et al. (Overview of ImageCLEFcaption 2017—the Image Caption Prediction and Concept Extraction Tasks to Understand Biomedical Images, CLEF working notes, CEUR, 2017).

For example, Biswall et al. (CLARA: Clinical Report Auto-completion, arXiv:2002.11701, 2020) proposed a system for clinical report auto-completion termed “CLARA”, which uses neural networks to learn embeddings from medical images and builds a prototype repository by indexing unique sentences in a large set of medical reports. Anchor words provided by a user are then used in combination with the image embeddings to retrieve template sentences, which are edited using a long short term memory (LSTM) network based encoder and decoder to generate a final sentence.

Boag et al. (Baselines for Chest X-Ray Report Generation, Proceedings of Machine Learning Research XX: 1-15, 2019 Machine Learning for Health (ML4H) at NeurIPS 2019) described a system for automated generation of free text reports from radiological images. The system includes a deep convolutional neural network (CNN), which is pre-trained using chest x-ray classification task, and a variety of language generation models, including a residual neural network (RNN). The RNN takes as input the output of the CNN, and uses a CNN encoder followed by a LSTM decoder trained to minimize the cross-entropy loss per token in the task of predicting the next word in the sentence.

Huang et al. (Multi-Attention and Incorporating Background Information Model for Chest X-Ray Image Report Generation, doi 10.1109/ACCESS.2019.2947134; 2019) described a tool for generating reports from x-ray image, which integrates the patient's background information with the image data. A CNN is used to generate image features, a RNN is used to generate sentence themes based on the image features, which are combined with the background information and used by another RNN to generate words based on the sentence theme and background information.

Many of these models used recurrent neural networks (LSTM is a type of RNN). Such models process text on a word-by-word basis and are not amenable to parallelization. Additionally, they often fail to learn long range dependencies from training text.

To exemplify the deficiency of RNNs and LSTMs when applied to medical images, U.S. Pat. No. 10,803,581 [Song] discloses the use of a convolutional neural network (CNN) and a recursive neural network (RNN) in a specific arrangement, i.e. connected in series, to automatically generate keywords only. Only keywords can be generated by Song for example: “intra cranial hemorrhage”, “no skull fracture”, “nasal”, “left frontal lobe”, “2.6×2.3”, and “soft tissue”. However Song does not automatically generate a complete diagnosis report 231 without significant human intervention. Column 11 lines 26 to 49 of Song disclose that the “user 105 may click on the keywords displayed in keywords be used for guiding the generated report based on keywords display area 223 to select one or more keywords that he/she 424 selected/added by a user , e.g. , using user interaction area would like to include in diagnosis report 231” and these users actions are described as “Select keywords from natural language description and display to the user” step S314 and “Receive a user interaction” step S316. Without such user interaction, Song does not disclose a process where a diagnosis report 231 can be generated without user selected keywords. Therefore Song is unable to generate a complete diagnosis report when provided only with a medical image 402 because it requires user input at steps S314 and S316 and this is a fundamental limitation of RNNs as they are limited in terms of the length of their generated sequence, and are not suitable for generating complete paragraphs or reports. Another limitation of Song is that it would be slow and unstable to train its RNN on long sequences. Although not explicitly mentioned in column 12 line 61 to column 13 line 21 where Song is describing how its model 400 is trained, it is known that the length of time to train an RNN is dependent on number of layers, number of neurons, batch size, epochs, learning rate, and dropout. RNNs are difficult to train properly due to the vanishing gradient and exploding gradient problems described in Bengio, Y., Simard, P., and Frasconi, P. (1994). “Learning long-term dependencies with gradient descent is difficult”, IEEE Transactions on Neural Networks, 5(2), 157-166. The gradients carry information used in the RNN, and when the gradient becomes too small, the parameter updates become insignificant. This makes the learning of long data sequences difficult. Long training time, poor performance, and bad accuracy are the major issues in gradient problems. The exploding gradient problem refers to the large increase in the norm of the gradient during training where the slope tends to grow exponentially instead of decaying. Such events are due to the explosion of the long term components and accumulation of large error gradients, which can grow exponentially more than short term ones, resulting in very large updates to the neural network model weights during the training process. The vanishing gradients problem refers to the opposite behaviour, when long term components go exponentially fast to norm 0, making it impossible for the model to learn correlation between temporally distant events. The problem with RNNs is that sequential computation inhibits parallelization, there is no explicit modelling of long- and short-range dependencies, and the distance between positions is linear.

Another limitation of Song is its complexity. LSTMs and GRUs (a variation on LSTMs) are described at column 12 line 37 as being required as solutions to the gradient problems described above with the use of RNNs, which adds complexity to Song's system. The reason LSTM layers are added in Song's system is to deal with the vanishing gradients problem of RNNs. Therefore Song's system requires multiple and various components such as CNNs, RNNs, LSTMs and GRUs to be present and interconnected in complex ways. Furthermore, due to Song's complexity, its system is unlikely to scale well on larger datasets and has poorer long-range dependency.

In addition, RNNs are particularly ill-suited for the generation of complete medical reports due to the strong long-term dependencies introduced by conditioning on the relevant medical images. In an RNN context, the images define the initial state of the RNN decoder. As the images are important when generating text throughout the report, not just at the beginning, the tendency of RNN generation to ‘forget’ long terms dependencies make them a particularly poor solution for the task.

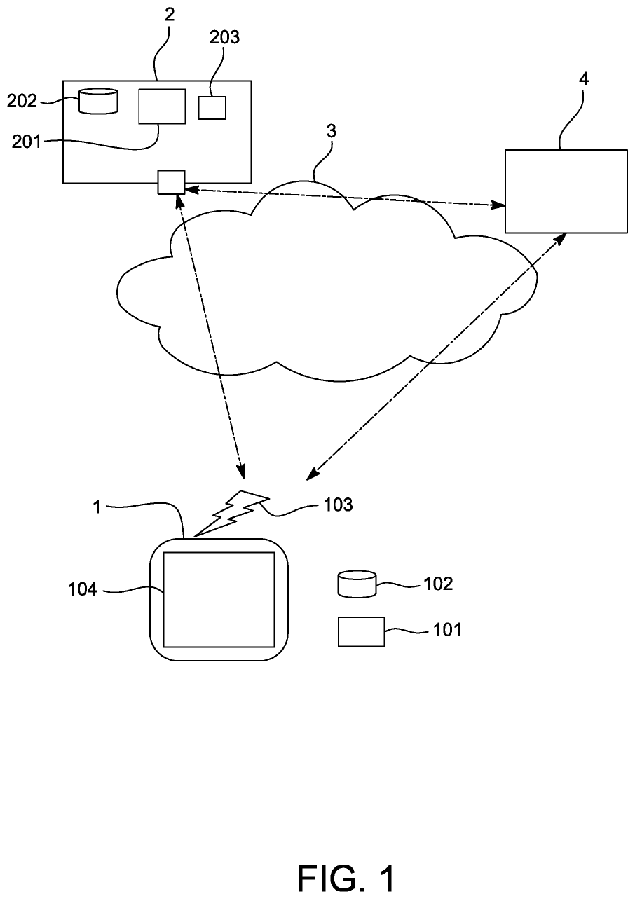

FIG. 1 is a block diagram illustrating a system 10 embodying the present invention.

A user (not shown) is provided with a first computing device (also referred to herein as “user computing device”). The user computing device may be a mobile computing device such as a mobile phone or any other device such as a personal computer 1. The first computing device 1 has at least one processor 101 and at least one memory 102 together providing at least one execution environment. The computing device 1 may also be equipped with means 103 to communicate with other elements of computing infrastructure, for example via the public interne 3. The first computing device 1 comprises a user interface 104 which typically includes a display. The display 104 may be a touch screen. Other types of user interfaces may be provided, such as e.g. a speaker, keyboard, one or more buttons (not shown), etc.

As also shown on FIG. 1, the system comprises a second computing device 2. The second computing device 2 may for example form part of a service provider computing system. The second computing device 2 typically comprises a processor 201 (which may in practice be implemented as a plurality of processor), which can be e.g. a server. The processor is interfaced to, or otherwise operably associated with a non-volatile memory/storage device 202, which may be a hard disk drive, and/or may include a solid-state non-volatile memory, such as ROM, flash memory, or the like. The processor is also interfaced to volatile storage 203, such as RAM, which contains program instructions and transient data relating to the operation of the server 201.

In a conventional configuration, the storage device 202 maintains known program and data content relevant to the normal operation of the server. For example, the storage device 202 may contain operating system programs and data, as well as other executable application software necessary for the intended functions of the server 201. The storage device 202 also contains program instructions which, when executed by the processor 201, instruct the server to perform operations relating to an embodiment of the present invention, such as are described in greater detail. In operation, instructions and data held on the storage device 202 are transferred to volatile memory 203 for execution on demand. In use, the volatile storage 203 contains a corresponding body of program instructions transferred from the storage device and configured to perform processing and other operations embodying features of the present invention.

The processor is also operably associated with a communications interface 204 in a conventional manner. The communications interface facilitates access to the data communications network 3.

Also shown on FIG. 1 is a secure system 4. The secure system 4 may be any computing or processing system requiring authentication of end-users prior to permitting access and/or the performance of transactions on behalf of those users. The secure system 4 is not described further here as the details of the secure system 4 used are not necessary for understanding how embodiments of the invention function and may be implemented.

The processor 201 may execute instructions (e.g. stored on the volatile storage 203) causing the processor to implement any of the steps of a method of generating text (e.g. captions) associated with medical images as described herein. For example, the processor 201 may receive medical images for which text is to be generated, from the user device 1. The processor 201 may store the images to be analysed in storage 203 and/or storage 201. The processor 201 may further execute instructions that cause it to implement all of the steps of any embodiment of a method of generating captions for medical images as described herein. In doing so, an output comprising one or more captions associated with the images may be produced. The processor 201 may store all or part of this output in storage 203 and/or storage 201. The processor may communicate at least part of this output (such as e.g. one or more captions) to the user device 1. In other embodiments, some or all of the steps of a method of generating text (e.g. captions) associated with medical images as described herein may be performed by the user device processor 101. In embodiments, the processor 201 may execute instructions (e.g. stored on the volatile storage 203) causing the processor to implement any of the steps of a method of providing a tool as described herein. For example, the processor may obtain training data from storage 202, and may use this data to train a hybrid model as described herein. The trained hybrid model may be stored in storage 201 and/or storage 203, and/or may be communicated to the user device 1 for local use. In embodiments, the user device processor 101 may execute instructions causing the processor to implement any of the steps of a method of providing a tool as described herein. For example, the user device processor 101 may obtain training data and/or provide training data to processor 201. The processor 101 may use the training data to train a hybrid model as described herein. The hybrid model may have been at least partially pre-trained, for example by processor 201, prior to re-training by processor 101. In this specification, terms such as ‘processor’, ‘computer’, and so forth, unless otherwise required by the context, should be understood as referring to a range of possible implementations of devices, apparatus and systems comprising a combination of hardware and software. This includes single-processor and multi-processor devices and apparatus, including portable devices, desktop computers, and various types of server systems, including cooperating hardware and software platforms that may be co-located or distributed. Hardware may include conventional personal computer architectures, or other general-purpose hardware platforms. Software may include commercially available operating system software in combination with various application and service programs. Alternatively, computing or processing platforms may comprise custom hardware and/or software architectures. For enhanced scalability, computing and processing systems may comprise cloud computing platforms, enabling physical hardware resources to be allocated dynamically in response to service demands. While all of these variations fall within the scope of the present invention, for ease of explanation and understanding the exemplary embodiments described herein are based upon single-processor general-purpose computing platforms, commonly available operating system platforms, and/or widely available consumer products, such as desktop PCs, notebook or laptop PCs, smartphones, tablet computers, and so forth.

In particular, the term ‘processing unit’ (or “computing device”) is used in this specification (including the claims) to refer to any suitable combination of hardware and software configured to perform a particular defined task, such as generating and transmitting data, receiving and processing data, or receiving and validating data. Such a processing unit may comprise an executable code module executing at a single location on a single processing device, or may comprise cooperating executable code modules executing in multiple locations and/or on multiple processing devices. For example, in some embodiments of the invention, processing may be performed entirely by code executing on a server, while in other embodiments corresponding processing may be performed cooperatively by code modules executing on the secure system 4 and server. For example, embodiments of the invention may employ application programming interface (API) code modules, installed at the secure system 4, or at another third-party system, configured to operate cooperatively with code modules executing on the server in order to provide the secure system 4 with useful services.

Software components embodying features of the invention may be developed using any suitable programming language, development environment, or combinations of languages and development environments, as will be familiar to persons skilled in the art of software engineering. For example, suitable software may be developed using the C programming language, the Java programming language, the C++ programming language, the Go programming language, and/or a range of languages suitable for implementation of network or web-based services, such as JavaScript, HTML, PHP, ASP, JSP, Ruby, Python, and so forth. These examples are not intended to be limiting, and it will be appreciated that convenient languages or development systems may be employed, in accordance with system requirements.

Broken lines shown in the system represent communications between an endpoint device, a secure system, and the server, embodying the present invention.

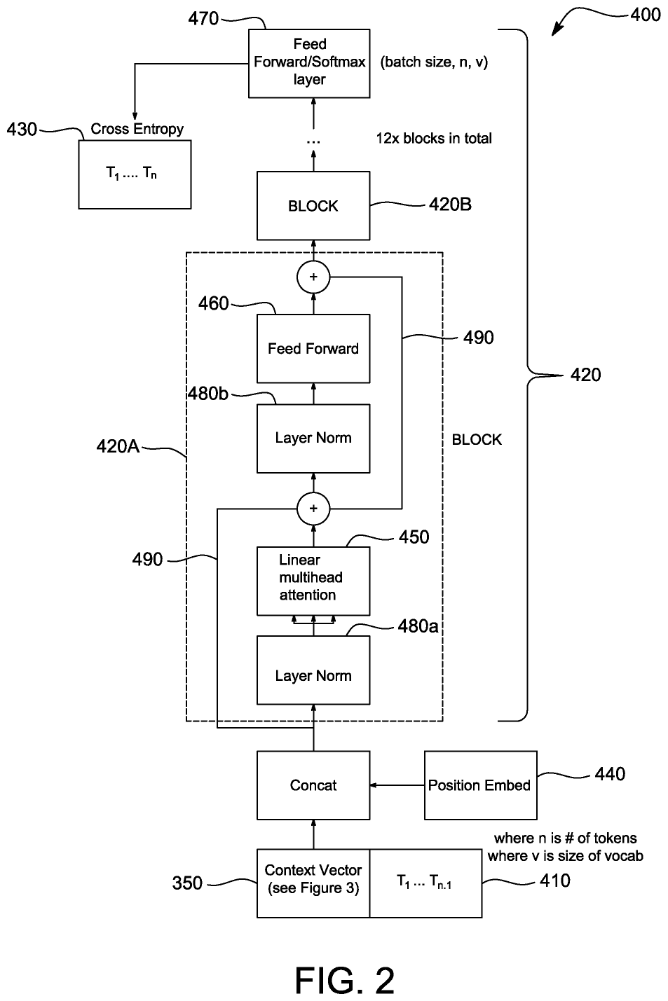

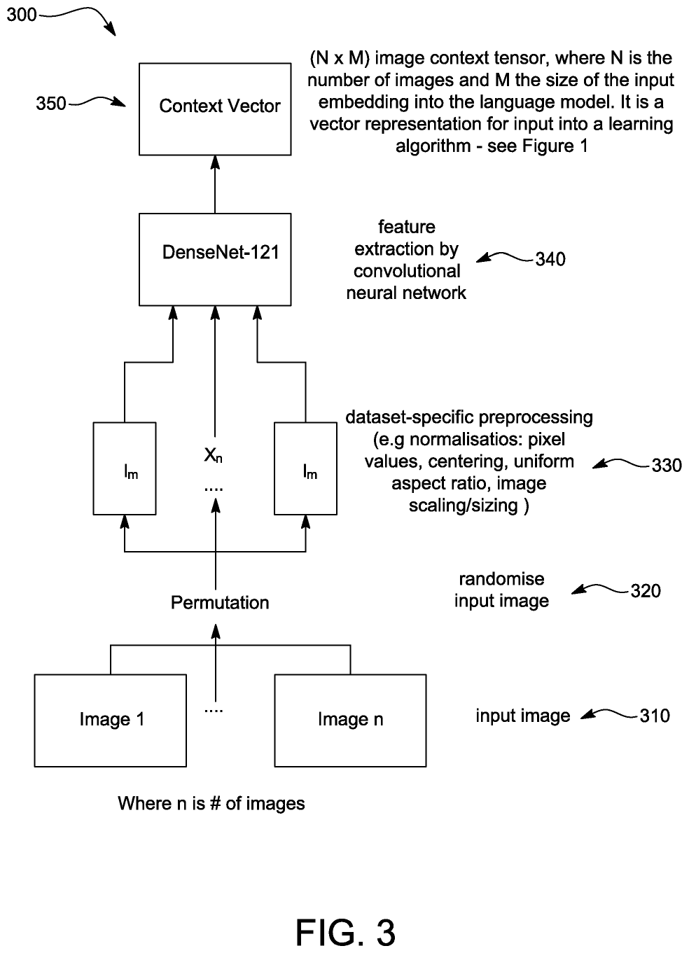

FIGS. 2 and 3 illustrate schematically the structure of a hybrid AI model according to embodiments of the present disclosure. A method of providing a tool according to embodiments of the invention will also be described by reference to the model displayed on FIGS. 2 and 3.

Within the context of the present disclosure, a “hybrid model” or “hybrid AI model” refers to a model that includes an image analysis component (typically a convolutional neural network, CNN) and a language processing component (typically a natural language processing (NLP) model, preferably a transformer-based model).

The transformer-based model benefits the system 4 during the decoding phase. Decoding with a transformer-based model is particularly beneficial for generating reports that are conditioned on one or more images (such as medical reports). As the images are provided as initial context at decoding time, and are possibly relevant at every step of the decoding process, it is important that the decoder architecture is capable of modelling these long-range image-text dependencies. The present inventors have recognised that transformers are particularly advantageous in this context as they are capable of attending to all previous context when generating text, with no recency bias. In contrast, RNNs struggle to maintain long term dependencies at decoding time.

The present inventors have recognised that transformer-based models are particularly advantageous in this context as they are significantly faster to train than RNNs. Transformer-based models are based on a self-attention mechanism, which is implemented purely through highly parallelisable and optimised matrix multiplication routines. In contrast RNNs are inherently sequential and must be unrolled, which inhibits parallelisation and increases train time.

The present inventors have recognised that transformer-based models are particularly advantageous in this context as they can advantageously scale to longer sequences. When training, the system 4 is configured to consume transformed chunks (e.g. 100 tokens) each time, after the words of the medical text are tokenised. At a system level, the transformer is a simple building block because the tokens are fed through this building block. A transformer-based model does not have recurrent state, which increases the maximum dimensionality.

The present inventors have recognised that transformer-based models are particularly advantageous in this context as they have improved long term dependency. The transformer-based model is indifferent to word order, but rather recognises the relationship between words. The transformer-based model can attend or focus on all previous tokens that have been generated.

The present inventors have recognised that transformer-based models are particularly advantageous in this context during decoding, as they allow the system 4 to attend to regions of the medical image that were responsible for generating specific words. Paying attention to these words in the medical report and this section of the image improves explainability of the system 4 and the predictions the model generates.

The model comprises an image processing component which is illustrated on FIG. 3. The image processing component 300 takes as input 310 a set of N images I_{1 . . . N}. The images can be any type of medical images including images of histopathology slides, radiology images, images of gross pathology specimens, MRI scans, PET scans, etc. Preferably, the images only include images of the same type, such as e.g. histopathology slides. Images of the same type may refer to images acquired using the same modalities, such as e.g. a digital microscope and histopathology stains, a standard digital camera with no microscope magnification, a digital x-ray machine. The images of the same type may have been acquired on separate machines, in separate locations, and may show different types of biological samples (including different tissues, body parts, etc.). In embodiments, the training images may be limited to images that show the same type of biological samples (e.g. gross pathology images of the same organ, histopathology slides acquired using the same stains and/or of the same tissue, etc.).

The order in which the images I_{1 . . . N} are input is optionally randomly permutated 320, and some or all of the images are optionally pre-processed 330. For example, the images may be pre-processed to normalise the pixel values across images in the training set of images, to change the aspect ratio to a common aspect ratio across the set (for example by letterboxing/reverse letterboxing, i.e. padding pixels on two opposite sides of an image), to scale and/or re-size the image (for example by stretching or zooming an image). Any pre-processing that is commonly applied to images for the purpose of feature detection may advantageously be used herein. The optionally permutated and/or pre-processed images are input into a convolutional neural network (CNN), which is trained using these images to perform visual feature extraction 340. The CNN is preferably a pre-trained model such as e.g. DenseNet-121 (Huang et al., “Densely Connected Convolutional Network”, 2016, arXiv:1608.06993; available at arxiv.org/abs/1608.06993). CNNs that have been pre-trained to perform image analysis tasks, for example using image databases such as ImageNet (Deng, J., Dong, W., Socher, R., Li, L. J., Li, K. and Fei-Fei, L., 2009, June. Imagenet: A large-scale hierarchical image database. In Computer Vision and Pattern Recognition, 2009. CVPR 2009. IEEE Conference on (pp. 248-255). Ieee), are widely available. For example, an implementation of DenseNet-121 for PyTorch is available at www.kaggle.com/pytorch/densenet121. Another pre-trained CNN may be used instead or in addition to this, such as e.g. ResNet5 (He, K., Zhang, X., Ren, S. and Sun, J., 2016. Deep residual learning for image recognition. In Proceedings of the IEEE conference on computer vision and pattern recognition (pp. 770-778)), AlexNet (Krizhevsky, Alex; Sutskever, Ilya; Hinton, Geoffrey E. (2017 May 24). “ImageNet classification with deep convolutional neural networks”. Communications of the ACM. 60 (6): 84-90. doi:1 0.1145/3065386), VGG (Simonyan, K. and Zisserman, A., 2014. Very deep convolutional networks for large-scale image recognition. arXiv preprint arXiv: 1409.1556), InceptionV3 (Szegedy, C., Vanhoucke, V., Ioffe, S., Shlens, J. and Wojna, Z., 2016. Rethinking the inception architecture for computer vision. In Proceedings of the IEEE conference on computer vision and pattern recognition (pp. 2818-2826)), etc. As the skilled person understands, such pre-trained networks can be partially retrained in a process called “transfer learning”. The parameters of the CNN (those that are re-trained) are preferably randomly initialised, and learned using input training data. A pre-trained model, such as e.g. densenet-121, may optionally be re-trained in multiple stages. For example, the pre-trained model may be re-trained using a large training data set of relevant images, such as e.g. medical images, prior to being provided to a user. The user may then perform a further re-training using a (typically smaller) set of their own training data. Such pre-training may be particular advantageous when the user has limited input data. Further training by a user may be particularly advantageous in that it enables the user to train the hybrid model using the user's choice of seed text. This may contribute to the tailoring of the text prediction to a user's requirements or preferences. In embodiments, the further re-training by a user may be performed using a subset of the images that were used to re-train the model, and one or more user-defined associated training seed text.

The image processing component produces as output 350 a (N×M) image context tensor, where N is the number of images and M is the size of the input embedding into the language model. In other words, for each of the N images, the CNN produces as output a vector of size M which represents the features identified in the image and is a used as input to the NLP component, which will be described further below by reference to FIG. 2. The NLP component 400 generates a caption for a single image (I₁, when N=1), or for a group of images (I_{1 . . . N}, when N>1), based at least in part on the output 350 of the image processing component (image context tensor) as will be described further below.

As shown on FIG. 2, seed text may optionally also be available and used to generate a further input 410 to the NLP component. When implementing a method of providing a tool comprising training a hybrid model as described herein, training images (input 310 of the image analysis component 300) and accompanying training seed text (from which further input 410 of the NLP component 400 is obtained) must be provided. Seed text may comprise any text associated with the images. For the purpose of training the NLP component, the seed text (referred to herein as training seed text) comprises text providing information about each image or each set of images in the training data. In other words, the training data comprises S groups of images I^{l}_{1 . . . N1} to I^{S}_{1 . . . NS}, each group comprising between 1 and N images, and associated seed text S¹ to S^{S}. Each group of training images is processed by the image processing component 300 to generate an image context vector 350, which is used in combination with the corresponding input 410 obtained from the training seed text to train the NLP component. The training seed text is preferably consistent in the sense that similar images are associated with text that has similar cognitive content. This increases the likelihood of the model learning correct associations between particular visual features and particular cognitive contents. For example, multiple training images showing Hematoxylin and eosin (H&E) stained pathology slides are preferably each associated with seed text that captures the “H&E stain” concept. Conversely, training seed text may not be consistent if it amongst the training seed text associated with multiple sets of images showing H&E stained pathology slides, some of the training seed text captures the “H&E stain” concept and some of the training seed text does not (e.g. because it erroneously describes the image as associated with another modality). Further, the training seed text is preferably informative in the sense that it summarises or otherwise describes the training images or relevant features thereof. As the skilled person understands, it is not a requirement that all training seed text be consistent with each other or even informative (or informative to the same extent). However, the performance of the tool may be negatively impacted by the lack of sufficient consistent and informative training seed text. The training seed text may be provided by one or more users (for example one or more users may at least partially manually annotate training images), and/or may be automatically sourced from one or more data stores as text associated with the training image data. For example, a training data set comprising images and associated captions may be used for training the model. Such data sets may be available from specialist data bases such as e.g. MIMIC-CXR (mimic-cxr.mit.edu/about/access/) which provides chest radiographs and associated free-text radiology reports (377,110 images corresponding to 227,835 radiographic studies performed at the Beth Isreal Deaconess Medical Center, Boston Mass.), CheXpert (stanfordmlgroup.github.io/competitions/chexpert/) which provides chest radiographs and associated radiology reports (224,316 chest radiographs of 65,420 patients performed at Stanford Hospital), etc. When using the model described herein, the use of seed text is optional and provides context for the automatically generated text. In such circumstances, when seed text is used it can be provided by a user, automatically sourced, or a combination of both. For example, seed text may be obtained from one or more locations that store text associated with the images, such as e.g. a clinical history file associated with the images. In a particular example, where the present invention is used to automatically generate a radiology report for one or more radiology image(s) (e.g. chest x-rays) of a patient, it may be advantageous for seed text to be provided comprising a patient's clinical history. Indeed, radiology reports are typically expected to include at least some elements of the patient's clinical history. The seed text (if available) is tokenized. The process of tokenisation splits the text into single units such as sentences, words or parts of words (sometimes referred to as “subwords”). Preferably, the tokeniser uses byte pair encoding. Using byte pair encoding, text is separated into individual characters and commonly occurring pairs of consecutive characters are merged to generate the vocabulary. This results in a vocabulary that contains subwords that may be of different sizes, striking a balance between character level encoding (which perform poorly on large data sets) and word based encoding (which poorly handles infrequent words). The model is associated with a vocabulary of size v, which represents the set of different tokens that are used by the model, and can be obtained using training seed text data (and optionally including any new seed text data). Preferably, each of the units (e.g. subwords) in the seed text forms part of the vocabulary. As the skilled person understands, the information content that is provided by the seed text is related to whether the seed text contains units that are part of the model's vocabulary. When all of the seed text is captured by tokens in the vocabulary, all can contribute to the output of the model. At the other extreme, when the seed text does not contain any units that are part of the vocabulary, then the model will produce an output essentially as if no seed text had been provided. The tokenised seed text is embedded into a (K×M) tensor, where K is the number of input tokens and M the size of the input embedding. Embedding involves the mapping of the vocabulary to real numbers such that a vector of numbers is obtained representing the tokenised seed text. In particular, embedding may be performed by means of a lookup table where each input token is mapped to a vector of size M. The values of this vector are parameters that is preferably optimized during training of the model. The seed text tensor forms the further input 410 of the NLP component. The image context tensor 350 (output of the image processing component) is prepended or appended to this (K×M) tensor 410 to form the input embedding. The size of the image context tensor 350 (N×M, where N is the number of images) is matched to the size of the seed text tensor 410 (K×M, where K is the number of input tokens derived from the seed text) in the sense that they both have a dimension of size M that is the size of the input embedding used by the transformer-based language model, which will be described below. Further, the image context tensor 350 is preferably prepended to the seed text tensor 410 when the language model is one that is trained by left to right conditional text generation, such as e.g. GPT-2. Indeed, such models learn to generate the subsequent word token by looking at the tokens to the left of it. In this scenario the image context has to be prepended in order for the model to have access to that information when generating the next word of the automatically generated text.

The input embedding (i.e. the result of the concatenation of the image features tensor, which form the “context” for the text, and the result of embedding of the tokenised text) is processed by the transformer-based language model 420 to produce predicted probabilities 430 for every possible token in the vocabulary at each step. In other words, the model uses the input embedding (350 and optionally 410) to produce as output 430 a probability for each possible token in the vocabulary (subwords), for each subsequent word (i.e. predicting the most likely next word given all preceding words in a text). At each run, the model 420 produces a probability 430 for each possible word token in the vocabulary, given the image(s) (image context tensor 350) and all preceding words provided in the seed text tensor (410). For the first run of the model, the seed text tensor 410 is either empty (if no seed text was provided), or captures any seed text that has been provided. One or more next words is/are produced by sampling from the vocabulary using the probabilities 430 provided by the model. The next word is then included in a new seed text tensor 410 and the model 420 (or a series of parallel models each using one of the next words generated by sampling from the vocabulary) is re-run to predict the subsequent word in the same way. Repeating the process in this way a number of times generated a body of text or a plurality of bodies of text. The process is repeated for a number of iterations i. The stop criteria for the process is when the model ceases improving on a held out validation set. This is referred to as early-stopping. The number of iterations i may be a parameter of the method, such as e.g. a parameter provided by a user or set to a default value. The language model 420 is a single stack architecture. In one form, the model may be trained by teaching it a language model, the probability distribution of possible sequences of words, in an unsupervised way. An attention mask may be used by adding a matrix that will “forbid” tokens (e.g. words) to attend to one another, for example, tokens to attend to other tokens later in the sequence. For example, when using a causal language model, the attention mask may be used in training and in predicting text for unseen images such that only previous words (tokens) are used at every step (to predict each new word/token). In other words, using a mask, it is possible to train the model by making each token in the sequence predict the next one, and to generate new sequences in an auto-regressive way. In another form, the model may be trained by masking a fixed proportion of tokens at random in a sequence (e.g. masking 15% of subword tokens) and trains the model to recover these masked words. This pre-trained model can then be fine-tuned on many language understanding tasks such as named entity recognition, question answering and text classification. This enables the model to be useful in other areas in addition to text generation. In a yet further form, the model may comprise an encoder-decoder architecture which further comprises a cross-attention layer, whose weights will be randomly initialized, and transforming the attention mask on the decoder input as a left-to-right mask adapted for generation tasks.

In one embodiment, the language model 420 is a pre-trained transformer based model, such as GPT-2 (github.com/openai/gpt-2 as described in Alec Radford, Jeffrey Wu, Rewon Child, David Luan, Dario Amodei & Ilya Sutskever, “Language Models are Unsupervised Multitask Learners”, 2019, available at cdn.openai.com/better-language-models/language_models_are_unsupervised_multitask_learners.pdf). The GPT-2 model is described in Polosukhin, Illia; Kaiser, Lukasz; Gomez, Aidan N.; Jones, Llion; Uszkoreit, Jakob; Parmar, Niki; Shazeer, Noam; Vaswani, Ashish (2017 Jun. 12). “Attention Is All You Need”. arXiv: 1706.03762) . The GPT-2 transformer-based model is particularly suitable for the task of text generation as this is the primary task for which it was optimised. In other embodiments, the language model 420 may be a pre-trained BERT model, as described in Jacob Devlin, Ming-Wei Chang, Kenton Lee, Kristina Toutanova, “BERT: Pre-training of Deep Bidirectional Transformers for Language Understanding”, 2018, arXiv: 1810.04805. In other embodiments, the language model may be a pre-trained T5 transformer-based model, as described in Colin Raffel, Noam Shazeer, Adam Roberts, Katherine Lee, Sharan Narang, Michael Matena, Yanqi Zhou, Wei Li, Peter J. Liu, “Exploring the Limits of Transfer Learning with a Unified Text-to-Text Transformer”, 2019, arXiv: 1910.10683.v2. A transformer is a deep learning model that is frequently used in the context of natural language processing. It includes a set of encoders and a set of decoders. Each encoder processes input vectors and produces encodings which contain information about connections between the inputs (i.e. which parts of the inputs are relevant to each other). Decoders perform the opposite task, taking as inputs the encodings of the encoders and generating an output sequence using the contextual information provided by the encodings. Each encoder and decoder uses an attention mechanism that weights the relevance of the previous latent states (inputs) according to a learned measure of relevancy to the current token. Each encoder includes a self-attention mechanism and a feed-forward neural network. As illustrated on FIG. 2, the first encoder takes as input positional information 440 and the input embedding (which as explained above contains both the context vector 350 from the image processing module and vectors 410 that represent the seed text obtained using a byte pair encoding tokeniser). The positional information 440 contains the information about the order of the tokens in the seed text. Each encoder comprises a self-attention mechanism (linear multihead attention) 450 which processes its inputs and weights their relevance to each other to generate a set of output encodings. The output encodings are then further processed individually through a feed-forward neural network 460. These processed output encodings are passed to the next encoder, as well as the decoders. The decoders include a self-attention mechanism, an attention mechanism over the encodings, and a feed-forward neural network. The attention mechanism uses information from the encodings generated by the encoders. The first decoder takes positional information and embeddings of the output sequence as its input, rather than encodings from the previous decoder. The last decoder is followed by a feedforward and softmax layer 470, which produce the output probabilities 430 over the vocabulary—given the images and the previous words (which may comprise initial seed text). The term “cross entropy” on FIG. 2 refers to the logarithm of the predicted probability of the next word token in the training data. The cross entropy loss may be use as a loss function to optimise for, i.e. minimising the cross-entropy loss will result in a model that maximises the likelihood of the model correctly predicting the next word in the training data. Both the encoders and decoders also contain layer normalisation steps 480a, 480b which normalise the weights to sum to 1. As illustrated on FIG. 2, each layer of the transformer (block) 420A, 420B has multiple attention heads, which capture the relevance of tokens to each other according to different definitions of relevance. As illustrated on FIG. 2, the model can contain multiple layers 420A, 420B, such as e.g. 12, each layer comprising an encoder or a decoder. For example, a total of 12 blocks (layers) may be provided, where the encoder is composed of a stack of 6 layers and the decoder is composed of a stack of 6 layers. In embodiments, more than 12 blocks may be used. Provided that sufficient training text is available to obtain a pre-trained model, the use of additional blocks may improve the performance of the model. In embodiments, fewer than 12 blocks may be used. Each block/layer 420A, 420B contains sublayers including: a linear multi-head attention layer 450, and a feedforward layer 460, separated by layer normalisation steps 480a, 480b. Also shown are residual connections 490 which propagate information between non-consecutive sublayers. The NLP component 400 is pre-trained in an unsupervised manner, and retrained in a supervised manner as will be explained further below.

The NLP component 400 and the image processing component 300 are jointly trained using training data comprising pairs of image/image sets and their associated text report/caption. A causal language modelling loss is used to jointly train the language model 420 and the CNN 340 in the image analysis component. Causal language models learn to predict the most likely next token in the sequence in a left-to-right direction. GPT-2 is a causal language model. In other embodiments, a masked language model is used, such as e.g. BERT. Such models learn to predict tokens that came before or after a previous token by randomly masking a proportion of the tokens (e.g. 15%). The primary measure of performance that is used to train the hybrid model is the perplexity. Perplexity measures how accurately the model is able to predict the next word token given the previous words and the image(s). In other words, perplexity quantifies how well a probability model predicts a sample (test set) by calculating the inverse probability of a test sentence normalised by the number of words in the sentence. A model that minimises perplexity maximises the probability of the test data.

The tool may advantageously be deployed as a library which contains a pre-trained model, for example a model that has been trained by processing image captions from histopathology images obtained from Open-I.

In addition to the default image captioning model, the tool supports further development by end users by allowing: Retraining on your own dataset; and Customization of model architecture, including the ability to change the language model from GPT2 (default) to other Transformer based architectures (for example, Bidirectional Encoder Representations from Transformers—BERT, T5, etc).

Additional data sources with paired medical imaging/text captions that can be used to generate pre-trained models include: Open-I, MIMIC-CXR, CheXpert and Learning to Cure.

When used to predict text associated with an image or a set of images, multiple instances of the hybrid model may be run (e.g. in parallel or successively), each of which will predict a different text. The multiple predictions may be provided to a user, who can for example select the most appropriate text. The multiple predictions may be ranked by probability (i.e. combining the probabilities of each of the words that has been sampled at each successive iteration of the NLP component 400, as explained above). Alternatively, a single prediction may be provided, such as e.g. that which has the highest probability.

### EXAMPLES

A first exemplary model of the tool is trained from a plurality of histopathology images and their associated text captions from journal articles, specifically haematoxylin and eosin stained microscopy images from Open-I (openi.nlm.nih.gov). The images were obtained using the search terms “hematoxylin OR eosin”, and 100,857 images were obtained as a result of this search. (100 examples of the images used are listed at openi.nlm.nih.gov/api/search?fields=c&it=xg%2Cmc&m=1&n=100&query=hematoxylin%20OR%20eosin).

The model as described above was trained using these images and the text in the “caption” field for each entry in the Open-I search results.

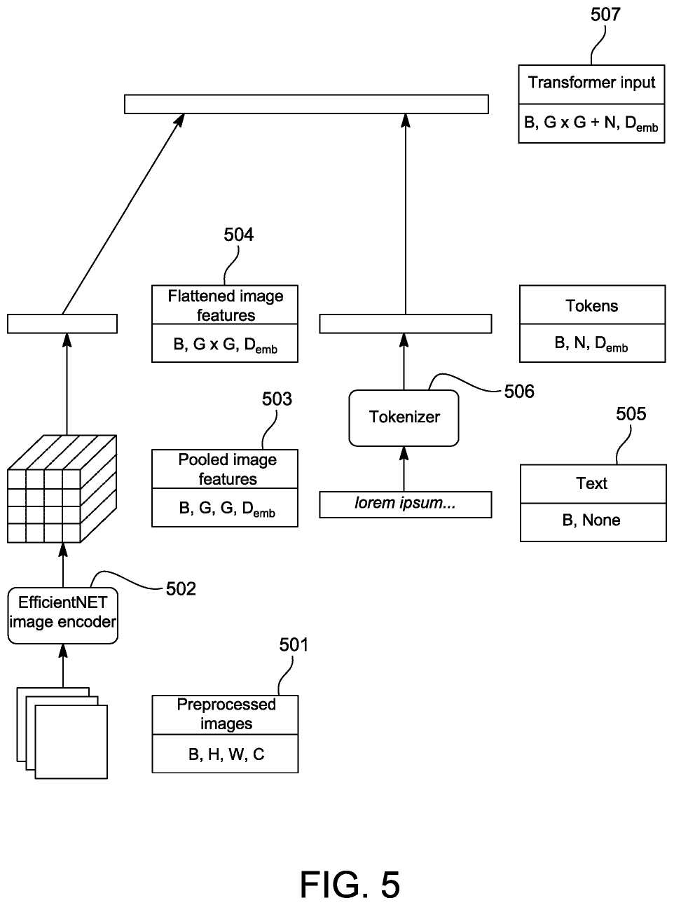

Referring to FIG. 5, the architecture of an image processing component of a hybrid AI model according to a second embodiment is depicted. Images are preprocessed 501 in a manner similar to the process depicted in FIG. 3. Images are included, where present, for both the current study and the last study for the same patient.

Next, the EfficientNet image encoder 502—efficientnet-b0 (without classification head), transforms the images into spatial-aware feature maps. The spatial-aware feature maps capture the global image contexts. A 1×1 convolution is used to ensure the feature-dimension of the map is the same as the dimensionality of the word embedding.

Next, for the pooled image features 503, dynamic mean- and max-pooling is used in order to reduce the feature maps to a fixed spatial size (in this example, G, G).

Next, for the flattened image features 504, the pooled image features are reshaped into a flat tensor.

Text 505 is supplied as raw text input, no preprocessing is required in this embodiment.

For the tokenizer 506, a Byte-Pair Encoding (BPE) tokenizer can pre-tokenize the words in the text by splitting the training data into words. After pre-tokenization, a set of unique words is created and the frequency of each word occurring in the training data is determined. The vocabulary is learned from the training data. BPE creates a base vocabulary consisting of all symbols that occur in the set of unique words and learns merge rules to form a new symbol from two symbols of the base vocabulary. Symbols which occur frequently together are merged. For instance the word “pathology” consists of two sub symbols “path” “ology”. If the word “pathology” occurs frequently enough in the text, then BPE will add an additional token to the vocabulary for “pathology”, so that the text doesn't have to be encoded as “path” +“ology”. It does so until the vocabulary has attained the desired vocabulary size. The desired vocabulary size is a hyperparameter to define before training the tokenizer, for example, it is 32000.

Lastly, for the transformer input 507, the flattened image features and the tokens are concatenated.

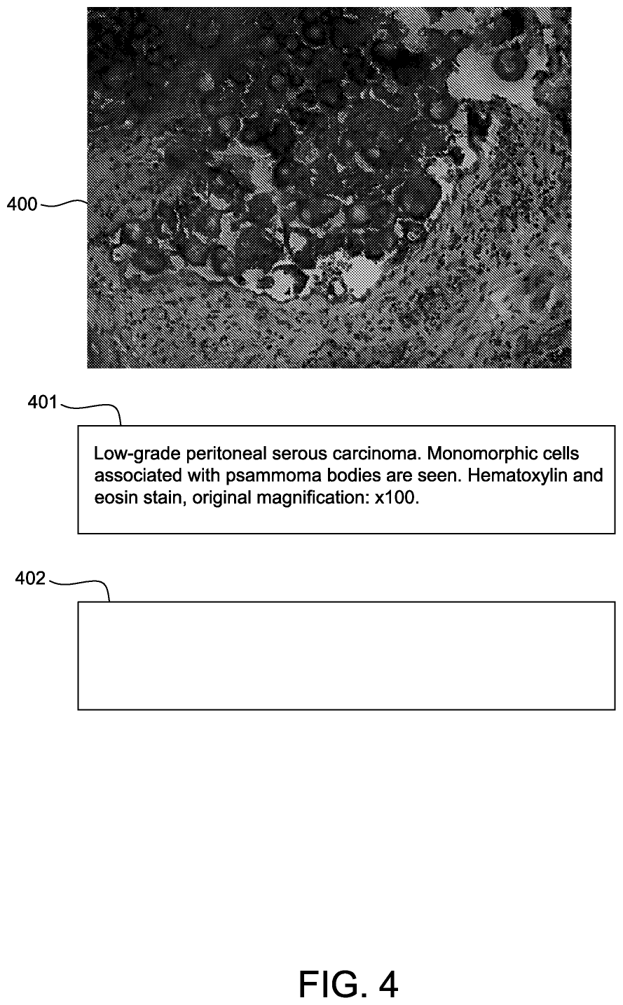

Referring to FIG. 4, an example is depicted illustrating a sample histopathology image 400 with an original associated caption 401. The generated captions 402 from the AI model are also illustrated and include:

Histological view showing multiple clear nucleoli in the dermis. There is intermingled hyalinized debris mixed with dense connective tissue, consistent with a high-grade thrombus but without associated chondrosarcoma, Hematoxylin and eosin stain, original magnification ×20.
Histology shows neoplastic cells with irregular nuclear membranes and nuclear hyperchromasia, suggestive of a leiomyomatous plasmacytoma [Hematoxylin &; Eosin, ×100]
Kidney sections obtained from dog 1: (a) Showing multiple hyphae with small round to pear shaped nests of atypical epithelioid cells, consistent with Paget's disease (hematoxylin and eosin, ×40); (b) High-powered view of a cyst with several islands of epithelioid cells (hematoxylin and eosin, ×400); (c) High-powered view of a cyst showing granulomas in the lumen; (d) Phagocytized germinal center with hyphae (immunostaining, ×400).
Pathology of the ureteral fibroma and connective tissue.
Hematoxylin-eosin staining; magnification, ×100.
Liver biopsy shows fibrosis (case 1) with a few inflammatory cells (Hematoxylin-eosin stain, 400×)
Photomicrograph shows neoplastic cells arranged in fascicles, stellate neoplastic cells with nuclear atypia and mitotic figures (hematoxylin and eosin stain, 3.5× magnification).
(Haematoxylin and Eosin, original magnification ×10) Microscopic view of a hematoxylin and Eosin-stained section of a glomerulus showing a glomerulus with several dilated capillary loops and normal capillary loops (arrows).
Pathological examination. Spindle cells (hematoxylin-eosin stain, original magnification ×25).
Photomicrograph of the specimen shows the neoplastic epithelioid cell tumor with fibroblastic stratification, lymphocytic infiltration, and numerous psammoma bodies (hematoxylin and eosin, ×100)
Hematoxylin &amp; Eosin stained section of the resected specimen demonstrating a hypercellular mesothelioma with intraductal papillary adenocarcinoma. Notes:

Performance on this dataset is limited due to the high variability of the text captions, as these are extracted from OpenI which in turn takes these from papers, all of which have different authors. As the image captions for OpenI do not have as defined structure, as image captions in academic papers are for illuminating something in the image in the context of the paper rather than a standalone summative description of the image, the training process does not work as well. Furthermore, text captions here are generated without seed text which removes any possible context for the model.

By contrast, significantly higher performances were obtained using seed text. Further, improved performance was also obtained in relation to the prediction of radiology reports, as such training data has a consistent structure.

In a second embodiment, the method comprises the use of fast transformers with linear attention. Fast transformers are defined as adopting a linear transformer model which enables reduction of memory requirements and linear scaling with respect to the context length. In other words, the quality of generated text from a fast transformer is comparable to a conventional transformer and is significantly more efficient in terms of inference time and memory. Another benefit of the method comprising the use of a fast transformer is to reduce the O(n²) computation complexity in standard key-value attention models used in a Generative Pre-trained Transformer (GPT)/standard self-attention mechanism to O(n) in both time and space with respect to sequence length, n denoting the sequence length. Fast transformers change the attention from conventional softmax attention to a feature map based on dot product attention. Attention implementations can include polynomial attention or RBF kernel attention. This enables longer sequence lengths and therefore additional tokens can be dedicated to the representation of the image. In the first embodiment, each image was converted to a single token or a small number of tokens via average and max pooling due to restrictions on the number of tokens. Average and max pooling in the first embodiment reduces the spatial resolution of feature maps and achieve spatial invariance to input distortions and translations either by propagating the average of all input values to the next layer or propagating the maximum value within a receptive field to the next layer, respectively. In the second embodiment, with the implementation of linear transformers (fast transformers), this second embodiment of the present invention is able to reduce the pooling size and increase the spatial resolution of the tokens per image compared to the first embodiment. This is likely to improve the ability of the language model to describe clinical findings in specific locations of a medical image. One feature of linear transformers which contributes to this improvement are the use of less memory “per token” as memory requirements scale linearly with the number of tokens (as opposed to quadratically for conventional transformers). This is due to the way that linear transformers attend to each token.

The fast transformers with linear attention configures the attention mechanism in conventional transformers in terms of a kernel function. The kernel function exploits information encoded in the inner product between all pairs of data items, and are successful partially because there is often an efficient method to compute inner product between very complex or even infinite dimensional vectors, providing a way to deal with nonlinear structures. The fast transformer converges smoothly and reaches a lower loss than Reformer (an efficient transformer proposed by Nikita Kitaev in January 2020) because of the lack of noise introduced by hashing. In particular, a fast transformer reaches comparable loss to a softmax transformer.

In addition, in the autoregressive context, a trained fast transformer model may have some properties of a recurrent neural network (RNN). This allows for the efficient training time and infinite context-window benefits of a traditional transformer, as well as the efficient decoding of an RNN model. In particular, because RNN decoding does not have to do a forward pass for every decode step, this formulation was demonstrated experimentally to improve decoding time by three orders of magnitude. In addition to reducing the time to generate text at deployment time, this also allows for decode steps to occur as part of the training loop, which allows for reinforcement learning based objective functions in addition to standard forward/backpropagation of loss. Reinforcement learning based objective functions (such as optimising directly for e.g. ROUGE scores) have been demonstrated to improve decoding quality, and improve training efficiency.

The use of a transformer-based model in the method of the present invention also improves explainability. When decoding, the transformer-based model enables regions of the medical image to be attended that were responsible for generating specific words. Additionally the use of a seed text within this transformer-based model enables users to explicitly pose clinical questions to the transformer-based model and receive relevant and explainable answers.

It should be appreciated that while particular embodiments and variations of the invention have been described herein, further modifications and alternatives will be apparent to persons skilled in the relevant arts. In particular, the examples are offered by way of illustrating the principles of the invention, and to provide a number of specific methods and arrangements for putting those principles into effect.

Accordingly, the described embodiments should be understood as being provided by way of example, for the purpose of teaching the general features and principles of the invention, but should not be understood as limiting the scope of the invention, which is as defined in the appended claims.

All references are incorporated herein in their entirety.

1. A computer implemented method for generating captions for medical images, the method comprising:
obtaining one or more medical images;
using an image processing component to process the one or more medical images, wherein the image processing component comprises a deep learning model that takes as input the one or more medical images and produces as an output an image feature tensor;
using a natural language processing component to generate a caption for the one or more medical images, wherein the natural language processing component comprises a transformer-based model that takes as input the image feature tensor from the image processing component and produces as output a probability for each word in a vocabulary.

2. The method of claim 1, wherein using a the natural language processing component to generate the caption for the one or more medical images comprises a first step which comprises using the transformer-based model to predict a probability for each word in the vocabulary and a second step which comprises sampling one or more words using the probabilities from the first step.

3. (canceled)

4. The method of claim 1, wherein the transformer-based model further takes as input an input tensor derived from a set of one or more words.

5-6. (canceled)

7. The method of claim 4, further comprising obtaining the input tensor by tokenising and embedding the set of one or more words.

8-10. (canceled)

11. The method of claim 1, wherein the transformer-based model is obtained by training a pre-trained GPT-2 model, a pre-trained BERT model or a pre-trained T5 model.

12. (canceled)

13. The method of claim 1, wherein the image processing component and the natural language processing component have been trained jointly to minimise at least one of the cross entropy loss and the perplexity of the predictions of the transformer-based model over a set of data.

14. The method of claim 1, further comprising receiving training data from a user and at least partially re-training the deep learning models in the image processing component and the transformer-based model in the natural language processing component using the training data.

15. The method of any claim 1, wherein the one or more medical images comprise multiple medical images and the method comprises generating a caption for the multiple medical images jointly, and wherein the multiple medical images are related to each other by sharing one or more features selected from: being associated with the same subject, being acquired using the same modality, showing the same pathology, showing the same organ or body part.

16. The method of claim 1, wherein the image processing component and the natural language processing component have been trained using training data comprising images that share one or more features with the one or more medical images, the one or more features being selected from: being associated with the same subject, being acquired using the same modality, showing the same pathology, showing the same organ or body part.

17. The method of claim 1, further comprising pre-processing the one or more medical images, by performing one or more steps selected from: randomly re-ordering the one or more medical images, normalising pixel values across the one or more medical images, changing the aspect ratio of one or more of the one or more medical images, scaling one or more of the one or more medical images, re-sizing one or more of the one or more medical images.

18. The method of claim 1, wherein the caption comprises free text.

19. The method of claim 1, wherein the one or more medical images are associated with a patient and the caption is a clinical report for the patient.

20. The method of claim 1, wherein the one or more medical images are selected from: histopathology images, radiography images, magnetic resonance images, ultrasound images, endoscopy images, positron emission tomography (PET) images, single-photon emission computed tomography (SPECT) images, and gross pathology images.

21. The method of claim 1, wherein the natural language processing component comprises a transformer-based model with a single stack architecture.

22. The method of claim 1, wherein the transformer-based model uses an attention mask that, is configured to forbid elements in an input tensor derived from a set of one or more words from attending to one another.

23. The method of claim 1, wherein the transformer-based model comprises one or more encoder and decoder blocks, each comprising a multi-head attention layer.

24. The method of claim 1, wherein the transformer-based model further takes as input a vector comprising information about a relative position of elements in an input tensor derived from a set of one or more words, wherein the relative position of the elements corresponds to an order of the one or more words in the set of one or more words from which the input tensor was derived.

25. (canceled)

26. The method of claim 7, wherein the input tensor has a size K×M, wherein M is the size of the embedding used by the transformer-based model and K is a number of tokens derived from the set of one or more words by tokenisation.

27. The method of claim 4, wherein the transformer-based model takes as input a tensor that comprises the image feature tensor pre-pended to the input tensor.

28-62. (canceled)

63. A system comprising:
at least one processor; and
at least one non-transitory computer readable medium containing instructions that, when executed by the at least one processor, cause the at least one processor to:
obtain one or more medical images;
use an image processing component to process the one or more medical images, wherein the image processing component comprises a deep learning model that takes as input the one or more medical images and produces as an output an image feature tensor;
use a natural language processing component to generate a caption for the one or more medical images, wherein the natural language processing component comprises a transformer-based model that takes as input the image feature tensor from the image processing component and produces as output a probability for each word in a vocabulary.

64-68. (canceled)
